(19) United States
(12) Patent Application Publication
(10) Pub. No.: US 2004/0062799 A1
(43) Pub. Date: Apr. 1, 2004

(54) Therapeutic composition and delivery system for administering drug

(76) Inventors: Atul D. Ayer, Palo Alto, CA (US); Michael A. Desjardin, Sunnyvale, CA (US); Paul M. Hwang, Mountain View, CA (US); Clark P. Allphin, Mountain View, CA (US)

Correspondence Address: PHILIP S. JOHNSON, JOHNSON & JOHNSON, ONE JOHNSON & JOHNSON PLAZA, NEW BRUNSWICK, NJ, 08933-7003, US

(21) Appl. No.: 10/661,168

(22) Filed: Sep. 12, 2003

Related U.S. Application Data
(63) Continuation of application No. 09/588,800, filed on Jun. 6, 2000, now abandoned.
Application No. 09/588,800 is a continuation of application No. 09/131,312, filed on Aug. 6, 1998, now abandoned.
(60) Provisional application No. 60/060,242, filed on Sep. 29, 1997.

(51) Int. Cl. A61K 9/20; A61K 9/24

(52) U.S. Cl. 424/465; 424/473

(57) ABSTRACT

A therapeutic composition comprising a drug and a polyitol, a dosage form comprising the therapeutic composition, and the method of using the therapeutic composition and the dosage form are disclosed for an indicated therapy.

### CROSS-REFERENCE TO RELATED APPLICATION

This application claims the benefits of provisional application U.S. Serial No. 60/060,242 filed Sep. 29, 1997 under 35 U.S.C. §119 (e).

### FIELD OF THE INVENTION

This invention pertains to both a novel therapeutic composition and to a novel delivery system. More particularly, the invention relates to (1) a therapeutic composition comprising a polyitol and a drug for administering to a patient, and, to (2) a delivery system comprising the therapeutic composition for delivering the therapeutic composition to a patient. The invention concerns also a method for administering a drug to a patient in need of therapy.

### BACKGROUND OF THE INVENTION

Delivery systems for administering a therapeutic composition to a patient in need of therapy are known to the medical and pharmaceutical arts. For example, in U.S. Pat. Nos. 3,845,770 issued to Theeuwes and Higuchi, and in U.S. Pat. No. 3,916,899 issued to the same patentees, a device is disclosed comprising a wall that surrounds a compartment containing a beneficial agent for delivery to a patient. The wall of the device is permeable to the passage of fluid and it comprises a passageway that is preformed or formed during use of the device for delivering the beneficial agent to the patient. The devices of these patents release the beneficial agent by fluid being imbibed through the wall into the device at a rate determined by the permeability of the wall and the osmotic pressure gradient across the wall. The fluid imbibed into the device mixes with the beneficial agent to form an aqueous media comprising the beneficial agent that is dispensed through the passageway from the device over time. The devices of these patents are extraordinarily effective for delivering a beneficial agent that is soluble and stable in aqueous and biological fluids and exhibit an osmotic pressure gradient across the wall against an external fluid. The devices of these patents are effective also for delivering a beneficial agent that is mixed with an osmotically effective solute soluble in fluid that exhibits an osmotic pressure gradient across the wall against an aqueous fluid.

A further advancement in the delivery art for dispensing a stable formulation is disclosed in U.S. Pat. No. 3,995,632 issued to Nakano, Higuchi, and Hussain. The dispenser disclosed in this patent dispenses a stable composition that absorbs heat and forms a dispensable melt. The melt is dispensed by a solution of magnesium sulfate increasing in volume and occupying the space originally occupied by the melt.

A quantum improvement in osmotic devices was presented to the pharmaceutical and medical dispensing arts by inventor Theeuwes in U.S. Pat. Nos. 4,111,202; 4,111,203; and 4,203,439. In these patents the delivery kinetics of the devices were enhanced for delivering a beneficial agent with different degrees of solubility in an aqueous-type fluid. The kinetics were improved by manufacturing the devices with a beneficial agent compartment separated by a film from an osmotic compartment. The devices deliver the beneficial agent by fluid being imbibed through the wall into the osmotic compartment to fill the compartment with fluid that acts as a driving force and thereby causes the film to move. The film moves against the beneficial agent compartment and the driving forces pushes the beneficial agent through a passageway from the device.

A pioneering advancement in osmotic delivery devices was made by Cortese and Theeuwes in U.S. Pat. No. 4,327,725 and by Wong, Barclay, Deters, and Theeuwes in U.S. Pat. No. 4,612,008. The devices disclosed in these patents comprise a semipermeable wall that surrounds a compartment. The compartment contains a beneficial agent formulation and a hydrogel. These devices operate by imbibing fluids into the compartment, wherein it contacts the beneficial agent formulation and forms a dispensable formulation, and wherein the imbibed fluid contacts the hydrogel causing it to expand and push the dispensable aqueous formulation from the device.

The delivery devices described in the above patents operate successfully for their intended use, and they can deliver many beneficial agents for their intended effects. Now, it has been observed their use can be limited because the devices lack the necessary elements to deliver beneficial agents that are sensitive or insensitive to fluids. The prior are sought to solve these limitation by using hydrogel swelling agents, to carry the beneficial agent from the device. These swelling agents, however, when contacted by an aqueous fluid often developed a swelling pressure so great they caused the wall of the device to rupture. Or, the swollen agent can entrap in its structure the beneficial agent and thereby make the beneficial agent unavailable for therapy.

It will be appreciated by those versed in the drug-dispensing art, in view of the above presentation, that a pressing need exists for a novel therapeutic composition and a delivery system essentially-free of the limitations associated with the prior art. It will be appreciated also, that if a therapeutic composition and a delivery system are provided essentially-free of a drug-swelling agent formulation, such a novel therapeutic composition and delivery system would have a positive value, and represent an advancement in the dispensing arts. Likewise, it will be self-evident, that a novel composition and delivery system, will have practical applications in the fields of human and veterinary medicine, and in the management of health.

### OBJECTS OF THE INVENTION

Accordingly, in view of the above presentation it is an immediate object of this invention to provide a therapeutic composition and a dosage from, which substantially overcome the deficiencies and omissions associated with the prior art.

Another object of the present invention is to provide a therapeutic composition that can administer a beneficial drug for the management of health.

Another object of the invention is to provide a therapeutic composition that can administer an aqueous insoluble to an aqueous soluble drug in an effective dose for a therapeutic benefit.

Another object of the invention is to provide a therapeutic composition for administering a dose of drug to a patient for establishing a drug level in the blood of the patient as a function of the dose administered by the therapeutic composition

Another object of the present invention is to provide a dosage form for administering a drug in a controlled-continuous-release rate to a patient for establishing an essentially-constant drug level in the blood as a function of a prolonged-release dosage form.

Another object of the present invention is to provide a dosage form that reduces and/or eliminates the unwanted influences of a gastrointestinal environment on the delivery of a drug in the gastrointestinal tract.

Another object of the present invention is to provide an improvement in a dosage form by administering a drug to a patient in need of drug therapy, wherein the improvement comprises delivering the drug from a dosage form in a continuous-release dose for predictable and improved therapy.

Another object of the present invention is to provide a method for administering a drug by orally administering the drug in a known dose per unit time over an extended time to a patient in need of therapy.

Another object of the invention is to provide drug delivery devices powered by osmotic energy for the controlled delivery of a therapeutically acceptable drug to an aqueous-biological environment of use.

These and other objects of this invention will be readily apparent to those skilled in the relevant are enabled by the disclosure herein.

### BRIEF DESCRIPTION OF THE DRAWINGS

In the drawing figures, which are not drawn to scale, but are set forth to illustrate various embodiments of the invention, the drawing figures are as follows:

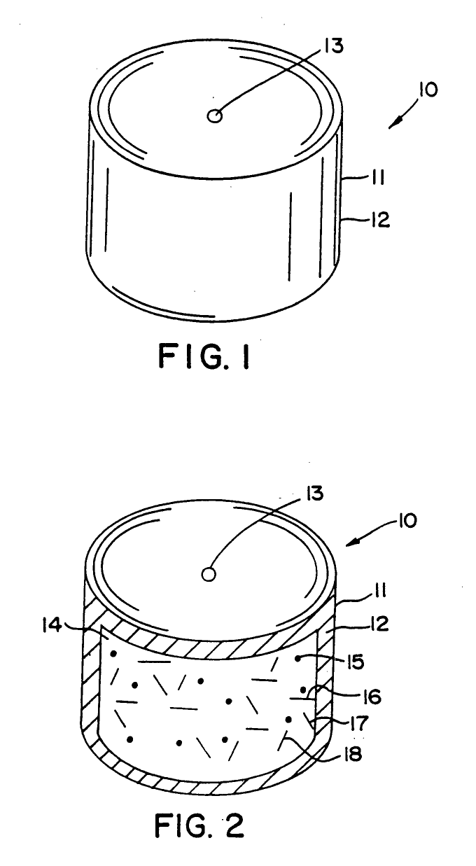

Drawing FIG. 1 as a general view of a dosage form, designed and shaped for oral administration for delivering a drug at a continuous-release rate over an extended time to a patient in need of therapy;

Drawing FIG. 2 is an opened view of the dosage form of FIG. 1, illustrating a dosage form comprising a single pharmaceutical composition comprising a drug and a pharmaceutically acceptable polyitol for delivering the pharmaceutical composition from the dosage form to a patient;

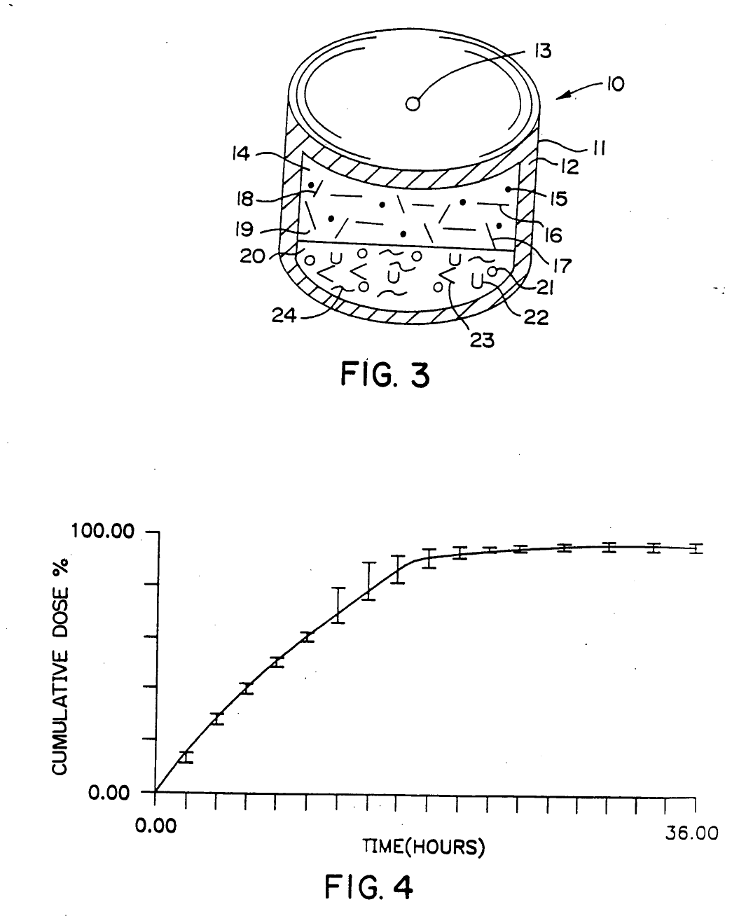

Drawing FIG. 3 is an opened view of drawing FIG. 1 illustrating a dosage form comprising a pharmaceutical composition comprising a drug and a polyitol and a displacement composition for pushing the pharmaceutical composition from the dosage form;

Drawing FIG. 4 illustrates the cumulative dose of drug delivery by a dosage form of this invention over time.

In the drawings, and in the specification, like parts on related figures are identified by like numbers. The terms appearing earlier in the specification and in the description of the drawings as well as embodiments thereof are further described in the specification.

### DETAILED DESCRIPTION OF THE DRAWINGS

Turning now to the drawing figures in detail, which drawing figures are examples of compositions and dosage forms provided by the invention, and which examples are not to be construed as limiting, one example of a dosage form comprising a composition is seen in drawing FIG. 1. In drawing FIG. 1, a dosage form 10 is seen comprising a body member 11 comprising a wall 12, this surrounds and forms an internal area, not visible in drawing FIG. 1. Drawing FIG. 1 comprises at least one exit 13 that connects the exterior of dosage form 10 with the interior of dosage form 10. The dosage form 10 of drawing FIG. 1 illustrates a controlled-release dosage form that delivers a drug over an extended time. The dosage form comprising the controlled-release properties provided by this invention is successful at maintaining therapeutic drug levels in blood or in body tissue. The terms blood and body tissue refer to human patients, zoo and farm animals. The dosage form provided by this invention makes available to the practice of medicine continuous-release, extended-release therapy. The phrase extended release embraces sustained release and prolonged release over up to a single day of therapy. The extended, prolonged and sustained release denotes a duration of drug delivery time over that achieved by conventional drug delivery forms such as tablets and capsules.

In drawing FIG. 2, dosage form 10 of drawing FIG. 1 is seen in opened-section. In drawing FIG. 2, dosage form 10, compress a body 11, a wall 12 that surrounds and defines an internal compartment 14. Internal compartment 14 communicates through exit 13 with the exterior of dosage form 10. Wall 12 of dosage form 10 comprises totally, or in as least a part a semipermeable composition. The semipermeable composition is permeable to the passage of an aqueous fluid, or a biological fluid present in the gastrointestinal tract, and it is impermeable to the passage of drug. Wall 12 is nontoxic and it maintains its physical and chemical integrity during the dispensing time of a drug. The phrase, maintains its physical and chemical integrity means wall 12 does not lose its structure and it does not undergo a major change during the dispensing of a drug.

Wall 12 comprises a composition that does not adversely affect an animal, a human, or components of the dosage form. Compositions for forming wall 12 are, in one embodiments, comprised a member selected from the group consisting a cellulose ester polymer, a cellulose ether polymer and a cellulose ester-ether polymer. These cellulosic polymers have a degree of substitution, DS on the anhydroglucose unit, from greater than 0 up to 3 inclusive. By “degree of substitution” is meant the average number of hydroxyl groups originally present on the anhydroglucose unit comprising the cellulose polymer that are replaced by a substituting group. Representative wall 12 polymers comprise a member selected from the group consisting of cellulose acylate, cellulose diacylate, cellulose triacylate, cellulose acetate, cellulose diacetate, cellulose triacetate, mono-, di- and tricellulose alkanylates, mono-, and di- and tricellulose alkinylates. Exemplary polymers include cellulose acetate having a DS of up to 1 and an acetyl content of up to 31%; cellulose acetate having a DS of 1 to 2 and any acetyl content of 21 to 35%; cellulose acetate having a DS of 2 to 3 and an acetyl content of 35 to 44.8%; and the like. More specific cellulosic polymers comprise cellulose propionate having a DS of 1.8, a propyl content of 39.2 to 45% and a hydroxyl content of 2.8 to 5.4; cellulose acetate butyrate having a DS of 1.8, an acetyl content of 13 to 15% and a butyryl content of 34 to 39%; cellulose acetate butyrate having a acetyl content of 2 to 29%, a butyryl content of 17% to 53% and a hydroxyl content of 0.5 to 4.7; cellulose triacylates having a DS of 2.9 to 3, such as cellulose trivalerate, cellulose trilaurate, cellulose tripalmitate, cellulose trisuccinate and cellulose trioctanoate; celluloses diacylate having a DS of 2.2 to 2.6, such as cellulose disuccinate, cellulose dipalmitate, cellulose dioctanoate, cellulose dipentanoate, co-esters of cellulose, such as cellulose acetate butyrate, and cellulose acetate propionate.

Additional semipermeable polymers comprise acetaldehyde dimethylcellulose acetate; cellulose acetate ethylcarbamate; cellulose acetate methylcarbamate; cellulose diacetate propylcarbamate; cellulose acetate diethylaminoacetate; semipermeable polyamide; semipermeable polyurethane; semipermeable sulfonated polystyrene; semipermeable crosslinked selective polymer formed by the coprecipitation of a polyanion and polycation, as disclosed in U.S. Pat. Nos. 3,173,876; 3,276,586; 3,541,005; 3,541,006 and 3,546,876; semipermeable polymers as disclosed by Loeb and Sourirajan in U.S. Pat. No. 3,133, 132; semipermeable, lightly crosslinked polystyrenes; semipermeable crosslinked poly (sodium styrene sulfonate); semipermeable cross-linked poly (vinylbenzyltrimethyl ammonium chloride); and semipermeable polymers possessing a fluid permeability of 2.5×10⁻⁸ to 5×10⁻² (cm²/hr·atm), expressed per atmosphere of hydrostatic or osmotic pressure difference across the semipermeable wall. The polymers are known to the polymer art in U.S. Pat. Nos. 3,845,770; 3,916,899 and 4,160,020; and in Handbook of Common Polymers, Scott, J. R. and W. J. Roff, 1971, CRC Press, Cleveland Ohio.

In drawing FIG. 2, internal compartment 14 comprises a single, homogenous composition. The composition comprises 0.5 wt % to 65 wt % of a drug, and 60 wt % to 99.5 wt % of a polyitol. The composition can comprise 0 wt % to 5 wt % of a lubricant, 0 wt % to 5 wt % of a binder, and 0 wt % to 3 wt % of a colorant. The total weight of all the compositional ingredients equals 100 wt %.

Dosage form 10 comprises in compartment 14 a therapeutic agent 15, represented by dots. The term therapeutic agent as used herein included medicines or drugs, nutrients, vitamins, food supplements, and other beneficial agents that provide a therapeutic or a benefit to animals, including a warm-blooded animal, humans, farm animals, and zoo animals. Representative of therapeutic agent 15 comprise vancomycin, phentolamine, valoxifene, cyclosporin, lisinopril, ondansetron, fluvoxamine, captopril, enalapril, amisulpride, imipramine, carbamazepine, famciclovir, clomipramine, penciclovir, pergolide, mesalazine, enitabas, talviraline, clozapine, nevirapine, zidoviudine, ganciclovir alendronic, imiquimod, naratriptan, sparflozacin, lamivudine, zidovudine, omeprazole, aiclovir, valaceclovir, oxcarbazepine, ganciclovir, amfebutamonc, cidofovir, doxazosin, ebastine, formoterol, moexipril, penciclovir, sertraline, spirapril, fenfluramine, dexfenfluramine, phentermine, fenphen, oxybutynin, felodipene, metoprolol, saquinavir, ritonavir, indinavir, and nelfinavir.

The therapeutic composition in compartment 14 comprises polyitol 16, represented by dashes. The polyitol comprises two or more HCOH groups. The polyitols are represented by a member selected from the group consisting of tetritols, pentitols, hexitols, heptitols, and octitols. The tetritols comprise a member selected from the group consisting of erythritol, mesoerythritol, D-threitol, and L-threitol; the pentitols comprise a member selected from the group consisting of ribitol, D-arabinitol, L-arabinitol, xylitol, mesoribitol, lyritol, and meso-xylitol; the hexitols are represented by a member selected from the group consisting of allitol, glucitol, mannitol, dulcitol, iditol, altritol, glactitol, talitol, maltitol and lactitol; the heptitols and octitols are represented by heptitol, and octitol.

The therapeutic composition comprises a lubricant 17 used during manufacture of the composition to prevent or reduce adhesion of the composition to the surface of dies and punches. The lubricants comprise calcium stearate, zinc stearate, magnesium stearate, magnesium oleate, calcium palmitate, sodium suberate, potassium laureate, stearic acid, salts of fatty acids, salts of alicyclic acids, salts of aromatic acids, oleic acid, palmitic acid, a mixture of a salt of a fatty, alicyclic or aromatic acid. The therapeutic composition can comprise a binder 18, represented by slanted dashes. The binder imparts cohesive qualities to the composition. Representative of materials for this invention useful as binders comprise a member selected from the group consisting of starch, gelatin, molasses, polyvinylpyrrolidone, methylcellulose and hydroxypropylmethylcellulose. The binder can be used as a solution, or in a dry form to prepare the therapeutic composition.

Drawing FIG. 3 depicts dosage form 10 comprising the therapeutic composition and a displacement composition for pushing the therapeutic composition from dosage form 10. The therapeutic composition comprises drug 15, polyitol 16, lubricant 17 and binder 18. The therapeutic composition is initially in contact with a displacement composition. The therapeutic composition can be classified a layer 19 and the displacement composition can be classified as layer 20, which layers 19 and 20 produce a bilayer core. The displacement composition comprises an osmopolymer 21 comprising 25 mg to 250 mg of a member selected from the group consisting of polyalkylene oxide of 1,500,000 to 8,500,000 weight-average molecular weight and carboxymethylcellulose of 175,000 to 6,000,000 weight average molecular weight. More specifically the polyalkylene oxide comprise polyethylene oxide and polypropylene oxide and the carboxymethylcellulose comprises alkali carboxymethylcellulose, sodium carboxymethylcellulose, and potassium carboxymethylcellulose. The displacement composition can comprise 0.5 mg to 50 mg of an osmagent 22. The osmagent 22 imbibes fluid into the displacement composition for cooperating with osmopolymer 21 for displacing the therapeutic composition from the dosage form. The osmagent 22 comprise a member selected from the group consisting of sodium chloride, potassuim chloride, lithium chloride, potassium acid phosphate, tartaric acid, citric acid, magnesium sulfate, magnesium chloride, urea, and a mixture of sodium chloride and urea. The displacement composition can comprise a colorant 23. The colorant 23 makes the dosage form more esthetic in appearance and it serves to identify the dosage form during manufacture and therapy. The colorants, comprise 0.00 to 4.5 mg, represented by FD &C Red No. 3; FD&C Red No 40; FD&C Yellow No. 5; FD&C Yellow No. 6; FD&C Blue No. 1; FD&C Blue No. 2; FD&C Green No. 3; and iron oxides including red ferric oxide and yellow ferric oxide. The displacement composition comprises 0.00 to 5 mg of a lubricant 24 selected from the group consisting of calcium stearate, magnesium stearate, zinc stearate, magnesium oleate, calcium palmitate, sodium laurate, stearic acid, oleic acid, palmitic acid, and a mixture of magnesium stearate and stearic acid.

The drawing figures provided above dictate the invention makes available a drug composition, a bilayer comprising a drug composition and a displacement layer, and a dosage form for administering a sustained release of drug. The invention provides for the drug composition and the displacement composition in a bilayer structure to be surrounded by a wall comprising a semipermeable composition, with a exit for delivering the drug to a human patient in need of therapy.

The expression “passageway” as used herein comprises means and methods suitable for the metered release of the therapeutic drug from the compartment of the dosage from. The exit means comprises at least one passageway including orifice, bore, aperture, pore, porous element, hollow fiber, capillary tube, porous overlay, or porous element that provides for the osmotic controlled release of drug. The passageway includes a material that erodes or is leached from the wall in a fluid environment of use to produce a dimensional passageway. Representative materials suitable for forming a passageway, or a multiplicity of passageways comprise a leachable poly(glycolic) acid or poly(lactic) acid polymer in the wall, a gelatinous filament, poly(vinyl alcohol), leachable polysaccharides, salts and oxides. A pore passageway, or more than one pore passageway, can be formed by leaching a leachable compound, such as sorbitol, from the wall. The passageway possesses controlled-release dimensions, such as round, triangular, square and elliptical for the metered release of drug from the dosage form. The dosage form can be constructed with one or more passageways in spaced apart relationship on a single surface or on more than one surface of the wall. The expression “fluid environment” denotes an aqueous or biological fluid as a human patient, including the gastrointestinal tract. Passageways and equipment for forming passageways are disclosed in U.S. Pat. Nos. 3,845,770; 3,916,899; 4,063,064; 4,088,864 and 4,816,263. Passageways formed by leaching are disclosed in U.S. Pat. Nos. 4,200,098 and 4,285,987.

### DESCRIPTION FOR MANUFACTURING THE COMPOSITION AND DOSAGE FROM OF THE INVENTION

The wall of the dosage form can be formed by using the air suspension procedure. This procedure consists in suspending and tumbling the composition or the layers in a current of air and wall-forming composition until a wall is applied to the drug forming compartment. The air suspension procedure is well suited for independently forming the wall. The air suspension procedure is described in U.S. Pat. No. 2,799,241; J. Am. Pharm. Assoc., Vol. 48, pp. 451459 (1959); and ibid., Vol. 49, pp. 82-84 (1960). The wall can be formed with a wall-forming composition in a Wurster® air suspension coater using an organic solvent, such as acetone-water cosolvent 90:10 (wt:wt) with 2.5 wt % to 7 wt % polymer solids. An Aeromatic® air suspension coater using, for example, a methylene dichloride methanol cosolvent comprising 87:13 (v:v) can be used for applying the wall. Other wall-forming techniques, such as pan coating, can be used for providing the dosage form. In the pan coating system wall forming compositions are deposited by successive spraying of the composition or the bilayered arrangement, accompanied by tumbling in a rotating pan. A larger volume of cosolvent can be used to reduce the concentration of polymer solids to produce a thinner wall. Finally, the wall of the coated compartments are laser or mechanically drilled, and then dried in a forced air or humidity over for 1 to 3 days or longer to free the solvent. Generally, the walls formed by these techniques have a thickness of 2 to 20 mils (0.051 to 0.510 mm) with a preferred thickness of 2 to 7 mils (0.076 to 0.180 mm).

The dosage form of the invention in another embodiment is manufactured by standard manufacturing techniques. For example, in one manufacture the beneficial drug and other ingredients comprising a therapeutic composition or comprising the first layer facing the exit means are blended, or the ingredients are blended then pressed, into a solid layer. The drug and other ingredients can be blended with a solvent and formed into a solid or semisolid formed by conventional methods such as ball-milling, calendering, stirring or roll-milling and then pressed into a selected shape. The drug layer posses dimensions that correspond to the internal dimensions of the area the drug layer is to occupy in the dosage from. Next, the drug layer is placed in contact with the displacement layer. The layering of the drug layer and the displacement layer can be fabricated by conventional press-layering techniques. The bilayers possess dimensions corresponding to the dimensions of the internal compartment or the dosage form. Finally, the two-layer compartment forming members are surrounded and coated with an outer wall. A passageway is laser drilled or mechanically drilled through the wall to contact the drug layer, with the dosage form optically oriented automatically by the laser equipment for forming the passageway on the preselected drug surface.

In another manufacture, the dosage from is manufactured by the wet granulation technique. In the wet granulation technique the drug and the ingredients comprising the first layer are blended using an organic or inorganic solvent, such as isopropyl alcohol-methylene dichloride 80:20 (v:v) as the granulation fluid. Other granulating fluid, such as water, isopropyl alcohol, or 100% denatured alcohol can be used for this purpose. The ingredients forming the first layer are individually passed through a 40 mesh or like screen and then thoroughly blended in a mixer. Next, other ingredients comprising the first layer are dissolved in a portion of the granulation fluid, such as the cosolvent described above. Then, the latter prepared wet blend is slowly added to the drug blend with continual mixing in the blender. The granulating fluid is added until a wet blend mass is produced, which wet mass is then forced through a 20 mesh or like screen onto oven trays. The blend is dried for 18 to 24 hours at 25° C. to 40° C. The dry granules are then screened with a 16 mesh or like screen. Next, a lubricant is passed through a 60 mesh or like screen and added to the dry screened granule blend. The granulation is put into milling jars and mixed on a jar mill for 2 to 10 minutes. The first and second layered compositions are pressed into a layered tablet, for example, in a Manesty® layer press.

Another manufacturing process that can be used for providing the drug and displacement compositions comprise blending their powdered ingredients 11 in a fluid bed granulator. After the powdered ingredients are dry blended in the granulator, a granulating fluid, for example, poly(vinylpyrrolidone) in a solvent, such as in water, is sprayed onto the respective powders. The coated powders are then dried in a granulator. This process coats the ingredients present therein while spraying the granulating fluid. After the granules are dried, a lubricant, such as stearic acid or magnesium stearate, is blended as above into the mixture. The granules are then pressed in the manner described above. In another embodiment, when the fluid be granulating process is used to manufacture the displacement layer, an antioxidant present in the polyalkylene oxide can be removed during the processing step. If antioxidant is desired, it can be added to the displacement layer, this can be accomplished during the fluid bed granulation described above.

The dosage form of this invention is manufactured in another embodiment by mixing a drug with composition-forming ingredients and pressing the composition into a solid layer possessing dimensions that correspond to the internal dimensions of the compartment space adjacent to a passageway. In another embodiment, the drug and other drug composition forming ingredients and a solvent are mixed into a solid, or semi-solid, by conventional methods such as ball-milling, calendering, stirring, or roll-milling, and then pressed into a preselected, layer-forming shape.

In the manufactures as presented above, the manufacture comprising a drug and a polyitol are placed in contact with the displacement layer, and the two layers are surrounded with a semipermeable wall. The layering of the drug composition and the second displacement composition can be accomplished by using a conventional two-layer tablet press technique. The wall can be applied by molding, spraying or dipping the pressed shapes into wall-forming materials. Another technique that can be used for applying the wall is the air-suspension coating procedure. This procedure consists in suspending and tumbling the two layers in a current of air until the wall forming composition surrounds the layers. Manufacturing procedures are described in Modern Plastics Encyclopedia. Vol. 46, pp. 62-70 (1969); and in Pharmaceutical Sciences, by Remington, 14th Ed., pp. 1626-1979 (1970) published by Mack Publishing Co., Easton, Pa. The dosage form can be manufactured by following the teaching the U.S. Pat. Nos. 4,327,725; 4,612,008; 4,783,337; 4,863,456; and 4,902,514.

Exemplary solvents suitable for manufacturing the wall, the composition layers and the dosage from include inert inorganic and organic solvents that do not adversely harm the materials, the wall, the layer, the composition and the drug. The solvents broadly include members selected from the group consisting of aqueous solvents, alcohols, ketones, esters, ethers, aliphatic hydrocarbons, halogenated solvents, cycloaliphatics, aromatics, heterocyclic solvents, and mixtures thereof. Typical solvents include acetone, diacetone, alcohol, methanol, ethanol, isopropyl alcohol, butyl alcohol, methyl acetate, ethyl acetate, isopropyl n-butyl acetate, methyl isobutyl ketone, methyl propyl ketone, n-hexane, n-heptane, ethylene glycol monoethyl ether, ethylene glycol monoethylacetate, methylene dichloride, ethylene dichloride, proplylene dichloride, chloroform, nitroethane, nitropropane, tetrachloroethane, ethyl ether, isopropyl ether, cyclohexane, cyclo-octane, toluene, naphtha, 1,4-dioxane, tetrahydrofuran, diglyme, aqueous and nonaqueous mixtures, such as acetone and water, acetone and methanol, acetone and ethyl alcohol, methylene dichloride and methanol, and ethylene dichloride and methanol.

### DETAILED DISCLOSURE OF EXAMPLES

The following examples are merely illustrative of the present invention and they should not be considered as limiting the scope of the invention in any way, as these examples and other equivalents thereof will become apparent to those versed in the art in the light of the present disclosure and the accompanying claims.

### Example 1

The therapeutic composition provided by the invention is prepared as follows: first, 2.4 g of oxybutynin hydrochloride, 42.6 g of mannitol, and 0.2 g of magnesium stearate are dry blended for 10 minutes in a 200 ml beaker, with mixing for 10 minutes with a stainless steel spatula. Next, the dry blend drug composition is compressed into a single layer tablet. 150 mg of the dry composition is compressed under a pressure head of two tons into a 9/32 inch (7.14 mm) diameter standard round tablet to provide the composition comprising a drug and polyitol.

### Example 2

A dosage form adapted, designed and shaped as a delivery device is manufactured as follows: first, 2.4 g of oxybutynin hydrochloride, 42.6 g of mannitol, and 0.2 g of magnesium stearate are dry blended for 10 minutes to produce a homogenous blend. Next, 150 mg of the drug composition is compressed under a pressure head of two tons into a 9/32″ (7.14 mm) diameter round tablet.

Next, a number of tablets are coated with a wall comprising a semipermeable polymer. The wall forming composition comprises 99% cellulose acetate having a 32.0% acetyl content, and 1% polyethylene glycol of 3350 molecular weight. The wall forming composition is dissolved in a acetone: water 88:12 (wt:wt) cosolvent to make a 5 wt % solids solution. The wall forming composition is sprayed onto the therapeutic tablets in a 24″ coater.

Then, one 25 mil (0.635 mm) exit passageway is drilled through the semipermeable wall to connect the dry, blended drug composition with the exterior of the dosage form. The residual cosolvent is removed by drying for 48 hours at 50° C. and 50% humidity. Next, the dosage forms are dried for 4 hours at 50° C. to remove excess moisture. The dosage forms produced by this manufacturer provide 5 wt % oxybutynin, 94.75 wt % mannitol, and 0.25 wt % magnesium stearate. The semipermeable wall comprises 99 wt % cellulose acetate comprising 32.0% acetyl content, and 1.0 wt % polyethylene of 3350 molecular weight. The dosage form comprises one exit passageway, 25 mil (0.635 mm) and has an oxybutynin mean release rate of 1.29 mg/hr.

### Example 3

The procedures of the above examples are followed for manufacturing a dosage form comprising a therapeutic composition weighing 154 mg and consisting of 3.25 wt % (4.92 mg) of oxybutynin hydrochloride, 89.50 wt % mannitol, 3.00 wt % hydroxypropylmethylcellulose of 9,200 molecular weight, 0.25 wt % magnesium stearate, and 4.00 wt % polyvinylpyrrolidone of 40,000 molecular weight. The therapeutic composition is surrounded by a wall weighing 14.3 mg and consisting of 48.8 wt % cellulose acetate comprising a 32% acetyl content, 46.20 wt % cellulose acetate comprising a 39.8% acetyl content, and 5.00 wt % polyethylene glycol of 3350 molecular weight. The dosage form comprises a 30 mil passageway. Accompanying FIG. 4 depicts the cumulative dose of oxybutynin hydrochloride released over 36 hours.

### Example 4

A therapeutic composition comprising a polyitol and a drug selected from the group consisting simivastatin, sumatriptan, doxazosin, amlodipine, azithromycin, lisinopril, and finasteride is prepared by following the above examples. The therapeutic composition is enveloped with a wall comprising a semipermeable composition and an exit in the wall for delivering the drug to a patient of need of therapy at a sustained-release rate over an extended time.

### Example 5

A dosage from adapted, designed as an elementary osmotic delivery device is manufactured as follows: First, the following ingredients are mixed in a beaker: oxybutynin chloride (9.75 grams), mannitol USP (268.5 grams), hydroxypropyl methyl cellulose of 9,200 molecular weight (9.0 grams), and polyvinylpyrrolidone of 40,00 molecular weight (112.0 grams). Then, 45 ml of anyhydrous ethanol is added to the mixture while stirring with a stainless steel spatula. The wet granulation is then passed through a 20-mesh box sieve, and dried at room temperature for approximately 16 hours. The dry granulation is then passed through a 20-mesh sieve once again.

Next, the granulation is placed in a jar, and magnesium stearate (0.75 grams) lubricant is added. This is blended for 90 seconds on a mechanical roller. Next, 154 mg of the drug granulation is compressed under a pressure head of 0.5 tons into a 9/32″ (7.14 mm) diameter standard round tablet.

The tablets are then coated with a semipermeable wall. The wall forming composition comprises 48.8% cellulose acetate having a 32.0% acetyl content, 46.2% cellulose acetate having a 39.8 acetyl content, and 5% polyethylene glycol having a molecular weight of 3350. The wall forming composition is dissolved in methylene chloride/methanol (80:20 wt:wt) cosolvent to make a 4% solids solution. The wall forming composition is sprayed onto the tablets in a 24″ coated to an average weight of 14.3 mg per system.

Next, one 25 mil (0.635 mm) exit passageway is drilled through the semipermeable wall. The residual solvent is removed by drying for 48 hours at 45° C. to 45° C. Next the osmotic dosage form are dried for 4 hours at 45° C. to remove excess moisture. The dosage form produced by this manufacture provides 3.25 wt % oxybutynin chloride, 89.5% mannitol possessing a 182 molecular weight, 3.0% hydroxymethylpropyl-cellulose, 4.0% polyvinylpyrrolidone, and 0.25% magnesium stearate. The semipermeable wall comprises 48.8 wt % cellulose acetate comprising a 32.0% acetyl content, 46.2% cellulose acetate having a 39.8% acetyl content, and 5.0 wt % polyethylene glycol with a molecular weight of 3350. The dosage form has one exit passageway, 25 mil (0.635 mm), and provides a mean release rate of 0.28 mg/hr between 0 and 14 hours.

### Method of Practicing the Invention

The invention pertains additionally to the use of the therapeutic composition, and the dosage form by providing a method for delivering a drug orally to a warm-blooded animal, including a human patient in need of therapy. The method comprises administering orally the therapeutic composition to a patient for therapy by admitting into the patient a therapeutic composition comprising a drug and a polyitol. The method comprises also admitting orally into a patient a dosage form comprising a semipermeable wall that surrounds a therapeutic composition comprising a dose of drug and a polyitol. The dosage form imbibes fluid through the semipermeable wall into the dosage form in response to the concentration gradient across the semipermeable wall. The therapeutic composition in the dosage form develops polyitol generated osmotic energy that causes the therapeutic composition to be administered through an exit in the wall over a prolonged period of time up to 30 hours to provide controlled and sustained therapy.

In summary, it will be appreciated that the present invention contributed to the art an unobvious dosage form that possesses practical utility, can administer a drug at a dose-metered release rate per unit time. While the invention has been described and pointed out in detail with reference to operative embodiments thereof, it will be understood by those skilled in the art that various changes, modifications, substitution and omissions can be made without departing from the spirit of the invention. It is intended, therefore, that the invention embrace those equivalents within the scope of the claims which follow.

We claim:

1. A therapeutic composition comprising 0.5 wt % to 65 wt % of a drug orally administrable to a patient and 60 wt % to 99.5 wt % of a polyitol pharmaceutically acceptable carrier for aiding in administering the drug to the patient.

2. A therapeutic composition comprising 0.5 wt % to 65 wt % of a drug orally administrable to a patient and 60 wt % to 99.5 wt % of a tetritol compatible with the drug as a pharmaceutically acceptable carrier for aiding in administering the drug to the patient.

3. A therapeutic composition comprising 0.5 wt % to 65 wt % of a drug orally administrable to a patient and 60 wt % to 99.5 wt % of a pentitol compatible with the drug as a pharmaceutically acceptable carrier for aiding in administering the drug to the patient.

4. A therapeutic composition comprising 0.5 wt % to 65 wt % of a drug orally administrable to a patient and 60 wt % to 99.5 wt % of a hexitol compatible with the drug as a pharmaceutically acceptable carrier for aiding in administering the drug to the patient.

5. A dosage form for delivering a drug to a patient, wherein the dosage form comprises 0.5 wt % to 65 wt % of a drug orally deliverable to a patient, and 65 wt % to 99.5 wt % of a polyitol compatible with the drug as a pharmaceutically acceptable carrier for the polyitol, for delivering the drug; a wall that surrounds the drug and the polyitol permeable to fluid and impermeable to the drug and polyitol; and an exit in the wall for delivering the drug from the dosage form to the patient.

6. A dosage form for delivering a drug to a patient, wherein the dosage form comprises 0.5 wt % to 65 wt % of a drug orally deliverable to a patient and 60 wt % to 99.5 wt % of a tetritol compatible with the drug, as a pharmaceutically carrier for delivering the drug; a wall that surrounds the drug and tetritol permeable to fluid and impermeable to drug and tetritol, and an exit in the wall for delivering the drug from the dosage from to the patient.

7. A dosage form for delivering a drug to a patient, wherein the dosage form comprises 0.5 wt % to 65 wt % of a drug orally deliverable to a patient and 60 wt % to 99.5 wt % of a pentitol compatible with the drug and serves as a pharmaceutically carrier for drug for delivering the drug; a wall that surrounds the drug and pentitol permeable to fluid and impermeable to drug and pentitol; and an exit in the wall for delivering the drug from the dosage from to the patient.

8. A dosage form for delivering a drug to a patient, wherein the dosage form comprises 0.5 wt % to 65 wt % of a drug orally deliverable to a patient, and 60 wt % to 99.5 wt % of a hexitol compatible with the drug and operates as a pharmaceutically carrier for delivering the drug; a wall that surrounds the drug and hexitol permeable to the passage or fluid and impermeable to drug and the hexitol; and, an exit in the wall for delivering the drug from the dosage form to the patient.

9. A dosage form for delivering oxybutynin to a patient, wherein the dosage form comprises 0.5 wt % to 65 wt % of oxybutynin orally deliverable to a patient, and 60 wt % to 99.5 wt % of a hexitol compatible with the oxybutynin that performs as a pharmaceutically acceptable carrier for oxybutynin; a wall that surrounds oxybutynin and hexitol and is permeable to the passage of fluid present in a patient and impermeable to the passage of oxybutynin and hexitol; and, an exit in the wall for delivering oxybutynin from the dosage from to the patient.

10. The dosage form according to claim 9, wherein the hexitol is mannitol.

11. The dosage form according to claim 9, wherein oxybutynin is present as oxybutynin hydrochloride.
